SEQUENCE LISTING (2 sequences)

SEQ ID NO: 1 - DNA, 20 bp, synthetic construct
  source 1..20: mol_type = other DNA
agagtttgatcctggctcag

SEQ ID NO: 2 - DNA, 17 bp, synthetic construct
  source 1..17: mol_type = other DNA
attaccgcggctgctgg

(19) United States
(12) Patent Application Publication
(10) Pub. No.: US 2023/0020958 A1
(43) Pub. Date: Jan. 19, 2023

(54) METHOD OF SELECTING A TREATMENT FOR AN MS PATIENT

(71) Applicants: Washington University, St. Louis, MO (US); University of Connecticut, FARMINGTON, CT (US); THE JACKSON LABORATORY, BAR HARBOR, ME (US)

(72) Inventors: Yanjiao Zhou, St. Louis, MO (US); Laura Piccio, Farmington, CT (US)

(73) Assignees: Washington University, St. Louis, MO (US); University of Connecticut, FARMINGTON, CT (US); THE JACKSON LABORATORY, BAR HARBOR, ME (US)

(21) Appl. No.: 17/813,561

(22) Filed: Jul. 19, 2022

Related U.S. Application Data
(60) Provisional application No. 63/223,525, filed on Jul. 19, 2021.

(51) Int. Cl. A61B 5/00; A61B 5/15; A61B 10/00

(52) U.S. Cl. CPC A61B 5/4076; A61B 5/15; A61B 10/0038

(57) ABSTRACT

Methods for identifying MS in a subject based on an analysis of the strength of the immune-microbial homeostatic relationship based on the immune profile and the gut microbiome profile are described. In addition, methods of identifying MS patients likely to seek disease-modifying treatment within six months based on an analysis of the relative abundance of Barnesiella spp. based on the gut microbiome profile are described.

### CROSS-REFERENCE TO RELATED APPLICATIONS

This application claims priority from U.S. Provisional Application Ser. No. 63/223,525 filed on Jul. 19, 2021, which is incorporated herein by reference in its entirety.

### STATEMENT REGARDING FEDERALLY SPONSORED RESEARCH OR DEVELOPMENT

Not applicable.

### Material Incorporated-by-Reference

The Sequence Listing, which is a part of the present disclosure, includes a computer-readable form comprising nucleotide and/or amino acid sequences of the present invention. The subject matter of the Sequence Listing is incorporated herein by reference in its entirety.

### FIELD OF THE DISCLOSURE

The present disclosure generally relates to methods of identifying MS in patients and the likelihood of seeking treatment based on combined data derived from blood and fecal samples.

### BACKGROUND OF THE DISCLOSURE

Multiple sclerosis (MS) is a chronic, autoimmune disease characterized by inflammation, demyelination, and axonal loss in the central nervous system (CNS). MS affects 2.5 million people worldwide, and imposes major burdens on individuals and society. The etiology of MS remains elusive, but has been postulated to result from host genetics and environmental factors. Dysregulation of immune response and abnormal metabolism in MS patients suggest that multiple systems are involved in its pathophysiology.

Gut bacterial communities modulate extra-intestinal immune and metabolic responses in experimental autoimmune encephalomyelitis (EAE), a commonly used mouse model of MS. Recent human studies have shown slight to moderate differences at the whole gut microbiome community level between MS patients and healthy controls. Intriguingly, specific microbes from MS patients and from controls can either adversely or beneficially influence EAE development, respectively. However, confounding factors such as demographics and diet that potentially influence the gut microbiome are not well addressed in previous microbiome studies related to MS, and their cross-sectional design is another common limitation. The significance of applying multi-omics in studying complex diseases was recently demonstrated. Given the multi-factorial nature of MS pathophysiology, a need exists for simultaneous, multi-system evaluations of host immune, metabolome, gut microbiome profiles, and diet over time.

### SUMMARY OF THE DISCLOSURE

In various aspects, methods for identifying MS patients and/or MS patients likely to seek disease-modifying treatments are disclosed herein.

In one aspect, a method for identifying MS in a subject is disclosed. The method includes obtaining a blood sample and a fecal sample from the subject, determining an immune profile based on the blood sample, determining a gut microbiome profile based on the fecal sample, and determining a strength of an immune-microbial homeostatic relationship based on the immune profile and the gut microbiome profile. The method further includes identifying MS in the subject if the strength of an immune-microbial homeostatic relationship falls below a threshold value.

In some aspects, the method may further include defining the threshold value based on a comparison of a first plurality of control strengths of immune-microbial homeostatic relationships of healthy controls and a second plurality of control strengths of immune-microbial homeostatic relationships of known MS patients.

In another aspect, a method of identifying an MS patient likely to seek disease-modifying treatment within six months is disclosed. The method includes obtaining a fecal sample from the subject, determining a gut microbiome profile based on the fecal sample, and determining a relative abundance of Barnesiella spp. based on the gut microbiome profile. The method further includes identifying an MS patient as likely to seek disease-modifying treatment within six months if the relative abundance of Barnesiella spp. falls above a threshold value.

In some aspects, the method may further include defining the threshold value based on a comparison of a first plurality of relative abundance of Barnesiella spp. of MS patients known to remain untreated for six months and a second plurality of relative abundance of Barnesiella spp. of MS patient known to seek disease-modifying treatment within six months.

Other objects and features will be in part apparent and in part pointed out hereinafter.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a block diagram schematically illustrating a system in accordance with one aspect of the disclosure.

FIG. 2 is a block diagram schematically illustrating a computing device in accordance with one aspect of the disclosure.

FIG. 3 is a block diagram schematically illustrating a remote or user computing device in accordance with one aspect of the disclosure.

FIG. 4 is a block diagram schematically illustrating a server system in accordance with one aspect of the disclosure.

FIG. 5 is a schematic of the MS study design. Stool and blood samples are collected from MS patients (n=30) and controls (n=25) at baseline and at six months after baseline. Stool samples are used for the gut microbiome characterization, and blood samples are used for immunophenotyping and global metabolome characterization. 4-day food diary is recorded to represent the regular dietary pattern of the study participants.

FIG. 6A is a plot of the principle component analysis (PCA) of the gut microbiome in MS patients and controls using baseline 16S rRNA data. The microbiome proportional data is subjected to log-ratio transformation. The resulting data is used for PCA analysis to view inter-participant variation in MS patients and controls.

FIG. 6B is a graph of the variance of baseline microbiome explained by clinical and demographic factors. Blood immune profile and BMI have significantly contribution to the microbiome variance, accounting for 4% and 3% of total variance. Diagnosis (MS vs controls) does not have significant impact on the microbiome variation

FIG. 6C is a set of graphs of taxa that are significantly different between MS patients and controls at baseline in 16S rRNA gene sequencing (FDR<0.05). Differential taxa are identified by DESeq2.

FIG. 6D is a set of graphs of taxa that are significantly different between MS patients and controls at baseline in metagenomic whole genome shotgun sequencing (FDR<0.05, DESeq2).

FIG. 6E is a set of graphs of baseline microbiome and initiation of DMTs in subsequent six months. Baseline microbiota that are significantly different in MS patients who received DMTs or did not receive DMTs in the preceding six months (FDR<0.05, DESeq2).

FIG. 7A is a Venn diagram to show the number of common and unique microbial genera from 16S rRNA gene and mWGS sequencing.

FIG. 7B is a graph of the relative abundance of top 20 genera detected by both mWGS and 16S.

FIG. 7C is a plot of the principle component analysis (PCA) of the baseline gut microbiome in MS patients and Controls using mWGS data. The microbiome proportional data is first subjected to log-ratio transformation. The resulting data is used for PCA analysis to view inter-participant variation in MS patients and Controls. PERMANOVA shows no statistical difference between MS patients and Controls in overall microbiome community.

FIG. 8 is a plot of the relative abundance of B. fragilis in baseline stool samples of MS patients and Controls.

FIG. 9 is a graph of the Spearman correlation of EDSS and BMI. Scatter plot shows a significant correlation between EDSS and BMI.

FIG. 10 is a set of graphs of short-chain fatty acid concentrations in MS patients and controls at baseline. Acetic acid, butyric acid and propionic acids in stool samples at baseline are determined by GC-MS and compared between MS patients and controls. No statistical significance is found between MS patients and controls by Wilcox sum rank test.

FIG. 11A is a plot of a PCA analysis of blood immune profile. MS patients and controls show a distinct immune profile, as indicated by the clear separation in first dimension of PCA. The overall immune profile in blood is statistically different between MS patients and controls (P=0.02, by PERMANOVA).

FIG. 11B is a set of plots of specific immune cell populations that differ between MS patients and Controls (FDR<0.05, Wilcoxon Rank Sum Test). MS patients exhibit an activated blood immune response, as indicated by an increase of proportion of many immune cell types.

FIG. 11C is a plot from PCA analysis of blood metabolome. The global metabolome profile between MS patients and controls are not statistically different.

FIG. 11D is a plot of a pathway enrichment analysis. x-axis is the enrichment (impact) factor, which is determined by the pathway topology analysis (the importance of a metabolite within a pathway). — log (P) in y-axis refers to negative natural logarithmic value of the original P value from statistical analysis of pathway difference between MS patients and controls. The size and color of each dot is positively correlated with the enrichment factor and p value, respectively.

FIG. 11E is a schematic of the multi-OMICS correlation by Mantel test. Mantel correlations are performed based on distance matrix of any two of OMICS datasets including the gut microbiome (Bray-Curtis distance), blood immune profile (Euclidian distance), metabolome (Euclidian distance) and food servings (Euclidian distance). Correlations were done for MS patients (blue color) and Controls (red color) separately. Rho (r) and p represent correlation co-efficiency and statistical significance of Mantel tests.

FIG. 11F is a schematic of the feature-feature correlations from multiple-OMICS in MS patients and Controls. 224 and 384 correlations were identified after multiple comparison adjustment for MS patients and Controls, respectively. The correlations were illustrated using Cytoscape. Red pentagons=Microbiome; Yellow squares=immune profile; Blue circles=blood metabolome; Green squares=food serving; Blue edges=negative correlations; Red edges=positive correlations.

FIG. 12 is a graph comparing Meat serving between MS patients and Controls at baseline, which are significantly different by Wilcox rank sum test before (P=0.01) and after (FDR=0.2) multiple testing correction.

FIG. 13 is a set of plots of distance correlation between multi-OMICS at baseline. Inter-participant distance is measured by Bray-Curtis dissimilarity for the gut microbiome, and by Euclidian distance for the immune, metabolome and food servings. Scattered plots are used to show the strength of correlations between any two distance matrices from the OMICS. R and p represent correlation coefficient and statistical significance of mantel tests.

FIG. 14A is an out-of-sample ROC curve of three classifiers in discriminate MS patients and Controls. The mean ROC curve was generated from 200 iterations of model validation for the microbiome. Means and 90% confidence intervals of AUC scores of ROC curve is listed at the bottom right for classification accuracy.

FIG. 14B is an out-of-sample ROC curve of three classifiers in discriminate MS patients and Controls. The mean ROC curve was generated from 200 iterations of model validation for the immune profile. Means and 90% confidence intervals of AUC scores of ROC curve is listed at the bottom right for classification accuracy.

FIG. 14C is an out-of-sample ROC curve of three classifiers in discriminate MS patients and Controls. The mean ROC curve was generated from 200 iterations of model validation for the blood metabolome. Means and 90% confidence intervals of AUC scores of ROC curve is listed at the bottom right for classification accuracy.

FIG. 14D is an out-of-sample ROC curve of three classifiers in discriminate MS patients and Controls. The mean ROC curve was generated from 200 iterations of model validation from a combination of the microbiome, immune profile, and blood metabolome. Means and 90% confidence intervals of AUC scores of ROC curve is listed at the bottom right for classification accuracy.

FIG. 14E is a graph of the top 20 important features from RF model. Eighteen blood metabolites (metabolite names start with mz) and two immune cell populations (T-bet+ memory T cell and memory Th17 cells) are ranked as the top 20 features of important from RF model in MS patients and controls classification.

FIG. 15 is a graph of the accuracy of model performance in classifying MS patients and controls as a function of number of features. Models with 20 features reaches to an accuracy>0.9 in discriminating MS patients and controls at baseline.

FIG. 16A is a set of plots of the temporal stability of the gut microbiome within six months in MS patients and controls. The stability of multi OMICS is evaluated by measuring the between-participant dissimilarity and within-participant dissimilarity (samples collected at both baseline and six months) of the gut microbiome (Bray-Curtis dissimilarity), immune profile (Euclidian distance), blood metabolome (Euclidian distance) in controls (n=22), MS patients who initiated DMTs (Treat_Yes, n=9), and MS patients who did not initiate DMTs (Treat_No, n=14). Difference of between- and within-participant dissimilarity is tested by Wilcox Rank Sum test.

FIG. 16B is a set of plots of the temporal stability of the blood immune profile within six months in MS patients and controls. The stability of multi OMICS is evaluated by measuring the between-participant dissimilarity and within-participant dissimilarity (samples collected at both baseline and six months) of the gut microbiome (Bray-Curtis dissimilarity), immune profile (Euclidian distance), blood metabolome (Euclidian distance) in controls (n=22), MS patients who initiated DMTs (Treat_Yes, n=9), and MS patients who did not initiate DMTs (Treat_No, n=14). Difference of between- and within-participant dissimilarity is tested by Wilcox Rank Sum test.

FIG. 16C is a set of plots of the temporal stability of the blood metabolome within six months in MS patients and controls. The stability of multi OMICS is evaluated by measuring the between-participant dissimilarity and within-participant dissimilarity (samples collected at both baseline and six months) of the gut microbiome (Bray-Curtis dissimilarity), immune profile (Euclidian distance), blood metabolome (Euclidian distance) in controls (n=22), MS patients who initiated DMTs (Treat_Yes, n=9), and MS patients who did not initiate DMTs (Treat_No, n=14). Difference of between- and within-participant dissimilarity is tested by Wilcox Rank Sum test.

FIG. 16D is a set of plots showing changes of the proportion of memory Th17 cells and GM-CSF+ memory T Cells from baseline to six months in MS patients received DMTs during study course.

Those of skill in the art will understand that the drawings, described below, are for illustrative purposes only. The drawings are not intended to limit the scope of the present teachings in any way.

### DETAILED DESCRIPTION

The present disclosure is based, at least in part, on the discovery that an analysis of combined data from gut microbiome, blood immune profile, circulating metabolomes, and diet in MS patients and healthy control individuals revealed differences in the relationships between these data from MS and control groups, in particular those data defining strengths of immune-microbial homeostatic relationships.

In various aspects, methods for identifying MS in a subject based on an analysis of the strength of the immune-microbial homeostatic relationship based on the immune profile and the gut microbiome profile are disclosed. In various other aspects, methods of identifying MS patients likely to seek disease-modifying treatment within six months based on an analysis of the relative abundance of Barnesiella spp. based on the gut microbiome profile are disclosed.

Additional descriptions of additional aspects of the disclosed methods are described in the examples below.

A control sample or a reference sample as described herein can be a sample from a healthy subject. A reference value can be used in place of a control or reference sample, which was previously obtained from a healthy subject or a group of healthy subjects. A control sample or a reference sample can also be a sample with a known amount of a detectable compound or a spiked sample.

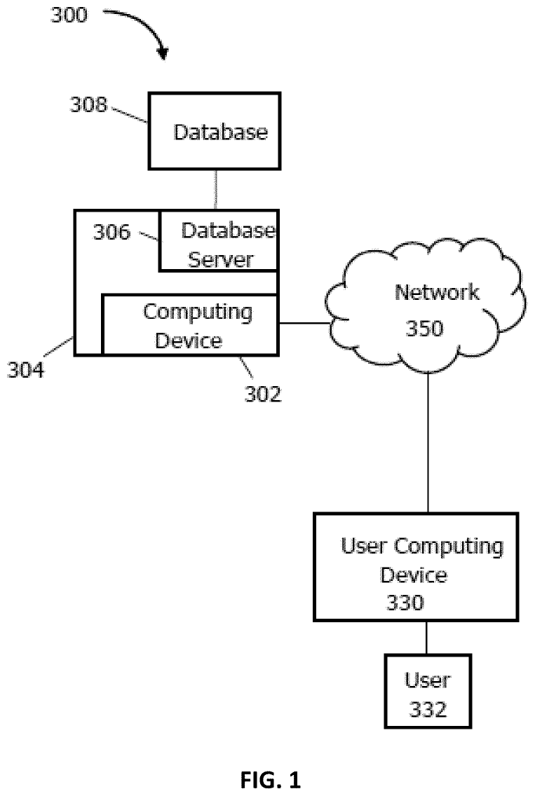

In various aspects, the disclosed method may be implemented using a computing system or computing device. FIG. 1 depicts a simplified block diagram of the system for implementing the computer-aided method described herein. As illustrated in FIG. 1, the computing device 300 may be configured to implement at least a portion of the tasks associated with the disclosed methods described herein. The computer system 300 may include a computing device 302. In one aspect, the computing device 302 is part of a server system 304, which also includes a database server 306. The computing device 302 is in communication with a database 308 through the database server 306. The computing device 302 is communicably coupled to a user computing device 330 through a network 350. The network 350 may be any network that allows local area or wide area communication between the devices. For example, the network 350 may allow communicative coupling to the Internet through at least one of many interfaces including, but not limited to, at least one of a network, such as the Internet, a local area network (LAN), a wide area network (WAN), an integrated services digital network (ISDN), a dial-up-connection, a digital subscriber line (DSL), a cellular phone connection, and a cable modem. The user computing device 330 may be any device capable of accessing the Internet including, but not limited to, a desktop computer, a laptop computer, a personal digital assistant (PDA), a cellular phone, a smartphone, a tablet, a phablet, wearable electronics, smartwatch, or other web-based connectable equipment or mobile devices.

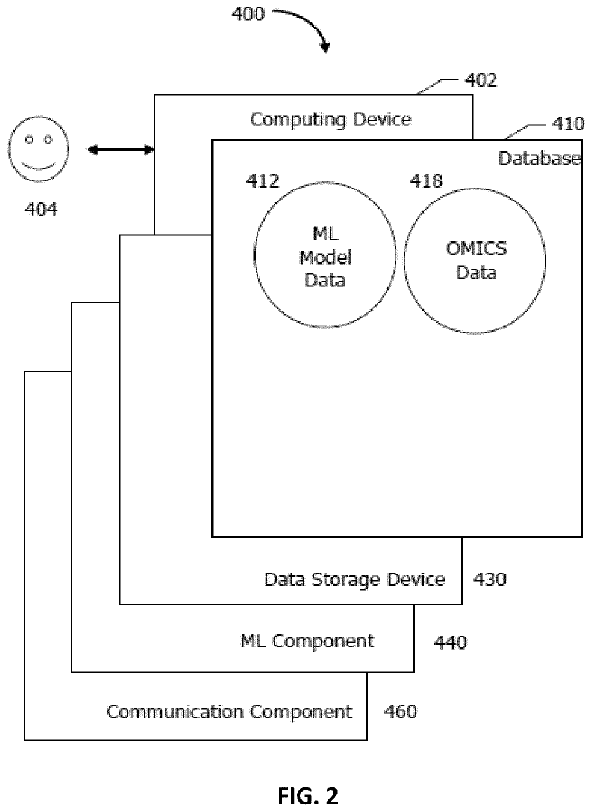

In other aspects, the computing device 302 is configured to perform a plurality of tasks associated with the disclosed computer-aided method of quantitative SPECT. FIG. 2 depicts a component configuration 400 of computing device 402, which includes database 410 along with other related computing components. In some aspects, computing device 402 is similar to computing device 302 (shown in FIG. 1). A user 404 may access components of computing device 402. In some aspects, database 410 is similar to database 308 (shown in FIG. 1).

In one aspect, database 410 includes OMICS data 418 and ML system model data 412. Non-limiting examples of suitable OMICS data 420 include any parameters indicative of the various measurements from genomics, metabolomics, proteinomics, and any other omics measurements as described herein. In one aspect, the ML model data 412 includes any values defining the parameters of the machine learning (ML) model configured to identify MS patients and/or identify a suitable treatment for an MS patient as described hererin.

Computing device 402 also includes a number of components that perform specific tasks. In the exemplary aspect, computing device 402 includes a data storage device 430, an ML component 440, and a communication component 460. ML component 440 is configured to implement a machine learning (ML) or artificial intelligence (AI) model used to identify MS patients and/or treatments for MS as described herein. Data storage device 430 is configured to store data received or generated by computing device 402, such as any of the data stored in database 410 or any outputs of processes implemented by any component of computing device 402.

The communication component 460 is configured to enable communications between computing device 402 and other devices (e.g. user computing device 330 shown in FIG. 1) over a network, such as a network 350 (shown in FIG. 1), or a plurality of network connections using predefined network protocols such as TCP/IP (Transmission Control Protocol/Internet Protocol).

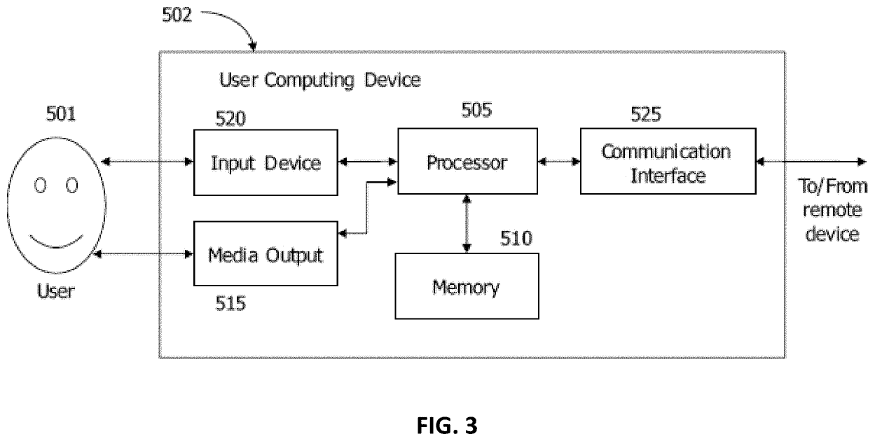

FIG. 3 depicts a configuration of a remote or user computing device 502, such as user computing device 330 (shown in FIG. 1). Computing device 502 may include a processor 505 for executing instructions. In some aspects, executable instructions may be stored in a memory area 510. Processor 505 may include one or more processing units (e.g., in a multi-core configuration). Memory area 510 may be any device allowing information such as executable instructions and/or other data to be stored and retrieved. Memory area 510 may include one or more computer-readable media.

Computing device 502 may also include at least one media output component 515 for presenting information to a user 501. Media output component 515 may be any component capable of conveying information to user 501. In some aspects, media output component 515 may include an output adapter, such as a video adapter and/or an audio adapter. An output adapter may be operatively coupled to processor 505 and operatively coupleable to an output device such as a display device (e.g., a liquid crystal display (LCD), organic light-emitting diode (OLED) display, cathode ray tube (CRT), or “electronic ink” display) or an audio output device (e.g., a speaker or headphones). In some aspects, media output component 515 may be configured to present an interactive user interface (e.g., a web browser or client application) to user 501.

In some aspects, computing device 502 may include an input device 520 for receiving input from user 501. Input device 520 may include, for example, a keyboard, a pointing device, a mouse, a stylus, a touch-sensitive panel (e.g., a touchpad or a touch screen), a camera, a gyroscope, an accelerometer, a position detector, and/or an audio input device. A single component such as a touch screen may function as both an output device of media output component 515 and input device 520.

Computing device 502 may also include a communication interface 525, which may be communicatively coupleable to a remote device. Communication interface 525 may include, for example, a wired or wireless network adapter or a wireless data transceiver for use with a mobile phone network (e.g., Global System for Mobile communications (GSM), 3G, 4G, or Bluetooth) or other mobile data network (e.g., Worldwide Interoperability for Microwave Access (WIMAX)).

Stored in memory area 510 are, for example, computer-readable instructions for providing a user interface to user 501 via media output component 515 and, optionally, receiving and processing input from input device 520. A user interface may include, among other possibilities, a web browser and client application. Web browsers enable users 501 to display and interact with media and other information typically embedded on a web page or a website from a web server. A client application allows users 501 to interact with a server application associated with, for example, a vendor or business.

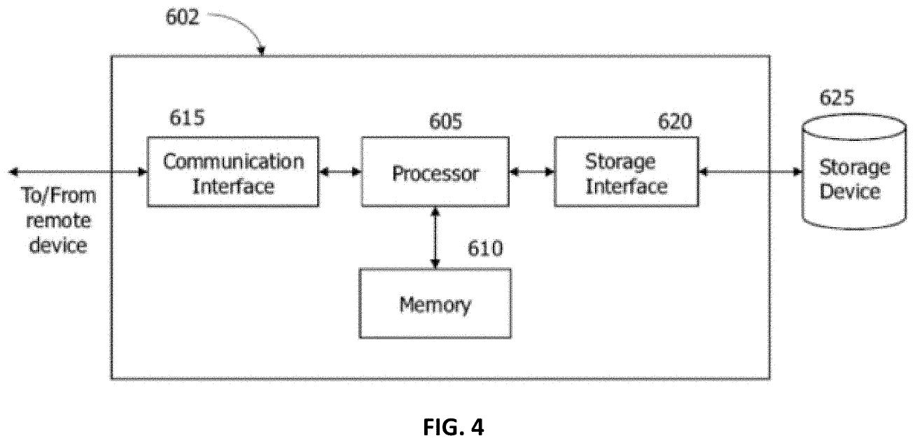

FIG. 4 illustrates an example configuration of a server system 602. Server system 602 may include, but is not limited to, database server 306 and computing device 302 (both shown in FIG. 1). In some aspects, server system 602 is similar to server system 304 (shown in FIG. 1). Server system 602 may include a processor 605 for executing instructions. Instructions may be stored in a memory area 625, for example. Processor 605 may include one or more processing units (e.g., in a multi-core configuration).

Processor 605 may be operatively coupled to a communication interface 615 such that server system 602 may be capable of communicating with a remote device such as user computing device 330 (shown in FIG. 1) or another server system 602. For example, communication interface 615 may receive requests from a user computing device 330 via a network 350 (shown in FIG. 1).

Processor 605 may also be operatively coupled to a storage device 625. Storage device 625 may be any computer-operated hardware suitable for storing and/or retrieving data. In some aspects, storage device 625 may be integrated into server system 602. For example, server system 602 may include one or more hard disk drives as storage device 625. In other aspects, storage device 625 may be external to server system 602 and may be accessed by a plurality of server systems 602. For example, storage device 625 may include multiple storage units such as hard disks or solid-state disks in a redundant array of inexpensive disks (RAID) configuration. Storage device 625 may include a storage area network (SAN) and/or a network attached storage (NAS) system.

In some aspects, processor 605 may be operatively coupled to storage device 625 via a storage interface 620. Storage interface 620 may be any component capable of providing processor 605 with access to storage device 625. Storage interface 620 may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing processor 605 with access to storage device 625.

Memory areas 510 (shown in FIG. 3) and 610 may include, but are not limited to, random access memory (RAM) such as dynamic RAM (DRAM) or static RAM (SRAM), read-only memory (ROM), erasable programmable read-only memory (EPROM), electrically erasable programmable read-only memory (EEPROM), and non-volatile RAM (NVRAM). The above memory types are examples only, and are thus not limiting as to the types of memory usable for the storage of a computer program.

The computer systems and computer-aided methods discussed herein may include additional, less, or alternate actions and/or functionalities, including those discussed elsewhere herein. The computer systems may include or be implemented via computer-executable instructions stored on non-transitory computer-readable media. The methods may be implemented via one or more local or remote processors, transceivers, servers, and/or sensors (such as processors, transceivers, servers, and/or sensors mounted on vehicle or mobile devices, or associated with smart infrastructure or remote servers), and/or via computer-executable instructions stored on non-transitory computer-readable media or medium.

The methods and algorithms of the disclosure may be enclosed in a controller or processor. Furthermore, methods and algorithms of the present disclosure, can be embodied as a computer implemented method or methods for performing such computer-implemented method or methods, and can also be embodied in the form of a tangible or non-transitory computer readable storage medium containing a computer program or other machine-readable instructions (herein “computer program”), wherein when the computer program is loaded into a computer or other processor (herein “computer”) and/or is executed by the computer, the computer becomes an apparatus for practicing the method or methods. Storage media for containing such computer program include, for example, floppy disks and diskettes, compact disk (CD)-ROMs (whether or not writeable), DVD digital disks, RAM and ROM memories, computer hard drives and back-up drives, external hard drives, “thumb” drives, and any other storage medium readable by a computer. The method or methods can also be embodied in the form of a computer program, for example, whether stored in a storage medium or transmitted over a transmission medium such as electrical conductors, fiber optics or other light conductors, or by electromagnetic radiation, wherein when the computer program is loaded into a computer and/or is executed by the computer, the computer becomes an apparatus for practicing the method or methods. The method or methods may be implemented on a general-purpose microprocessor or on a digital processor specifically configured to practice the process or processes. When a general-purpose microprocessor is employed, the computer program code configures the circuitry of the microprocessor to create specific logic circuit arrangements. Storage medium readable by a computer includes medium being readable by a computer per se or by another machine that reads the computer instructions for providing those instructions to a computer for controlling its operation. Such machines may include, for example, machines for reading the storage media mentioned above.

In some aspects, a computing device is configured to implement machine learning, such that the computing device “learns” to analyze, organize, and/or process data without being explicitly programmed. Machine learning may be implemented through machine learning (ML) methods and algorithms. In one aspect, a machine learning (ML) module is configured to implement ML methods and algorithms. In some aspects, ML methods and algorithms are applied to data inputs and generate machine learning (ML) outputs. Data inputs may include but are not limited to images or frames of a video, object characteristics, and object categorizations. Data inputs may further include sensor data, image data, video data, telematics data, authentication data, authorization data, security data, mobile device data, geolocation information, transaction data, personal identification data, financial data, usage data, weather pattern data, “big data” sets, and/or user preference data. ML outputs may include but are not limited to: a tracked shape output, categorization of an object, categorization of a region within a medical image (segmentation), categorization of a type of motion, a diagnosis based on motion of an object, motion analysis of an object, and trained model parameters ML outputs may further include: speech recognition, image or video recognition, medical diagnoses, statistical or financial models, autonomous vehicle decision-making models, robotics behavior modeling, fraud detection analysis, user recommendations and personalization, game AI, skill acquisition, targeted marketing, big data visualization, weather forecasting, and/or information extracted about a computer device, a user, a home, a vehicle, or a party of a transaction. In some aspects, data inputs may include certain ML outputs.

In some aspects, at least one of a plurality of ML methods and algorithms may be applied, which may include but are not limited to: genetic algorithms, linear or logistic regressions, instance-based algorithms, regularization algorithms, decision trees, Bayesian networks, cluster analysis, association rule learning, artificial neural networks, deep learning, dimensionality reduction, and support vector machines. In various aspects, the implemented ML methods and algorithms are directed toward at least one of a plurality of categorizations of machine learning, such as supervised learning, unsupervised learning, adversarial learning, and reinforcement learning.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### Examples

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

Multi-Omics of the Host-Gut Microbiome Dynamics in Multiple Sclerosis

Results

Baseline Characteristics of the Study Population

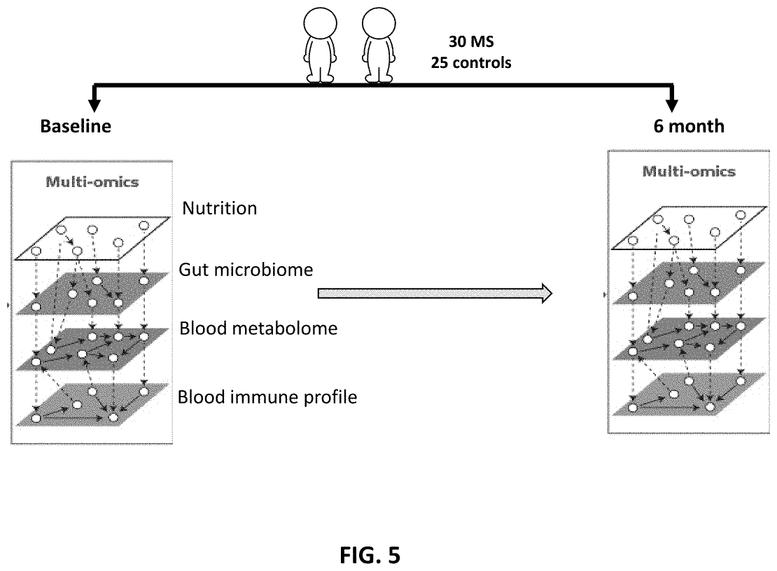

Thirty MS patients and 25 controls were recruited for the study. Stool and blood were collected at entry and six months later for gut microbiome, blood metabolome and blood immune cell analyses (FIG. 5). A 4-day food diary was also recorded to provide qualitative dietary information. Of the 30 MS patients, 24 were diagnosed with relapsing-remitting (RRMS), 2 with primary progressive (PPMS), 2 with secondary progressive (SPMS), and 2 with clinical isolated syndrome; mean disease duration at study entry was 6.4 (SD 1.5) years. At entry (baseline), no MS patient was in active relapse or had received any disease-modifying therapies (DMTs) in the previous 3 months. Characteristics of the participants at baseline are summarized in Table 1 in Appendix C. Except for the MS group taking more supplements (P=0.03, chi-square) and using more tobacco (P<0.0005, chi-square test), the characteristics of the control and MS groups did not differ significantly.

Overall Gut Microbiota Profile in MS Patients and Controls and Factors Underlying Microbial Variation

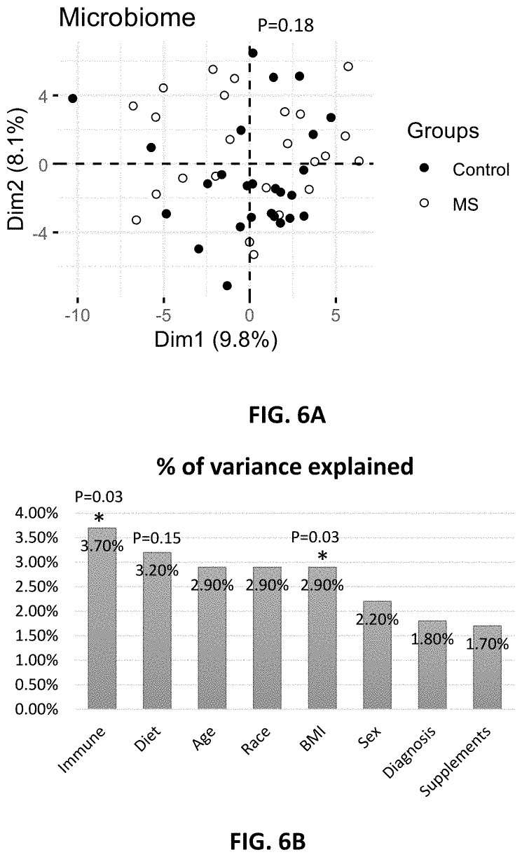

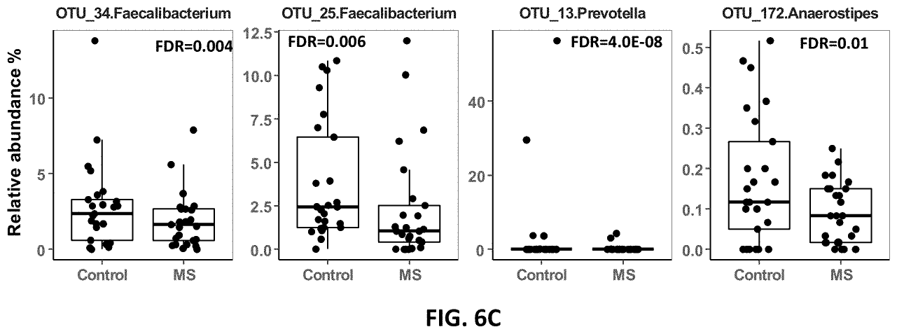

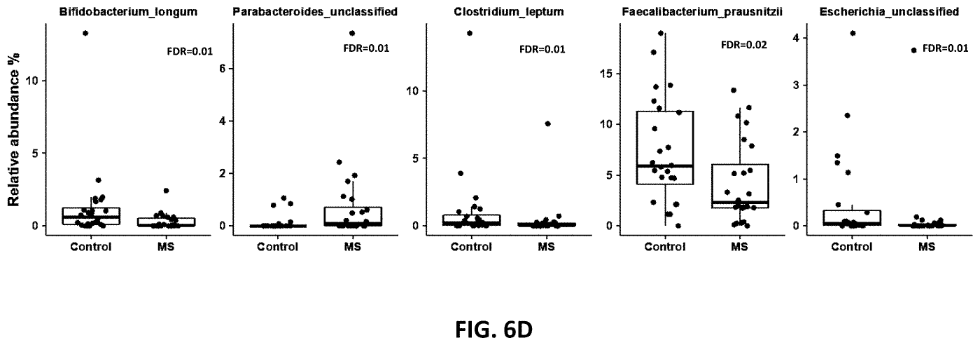

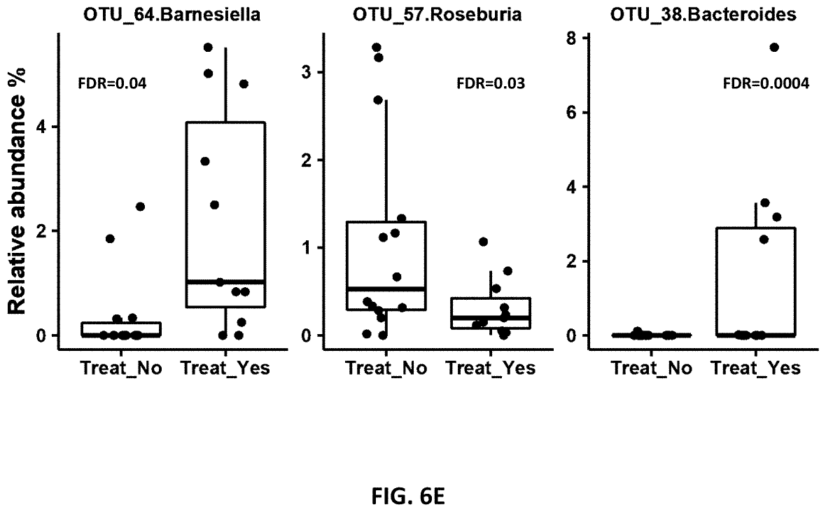

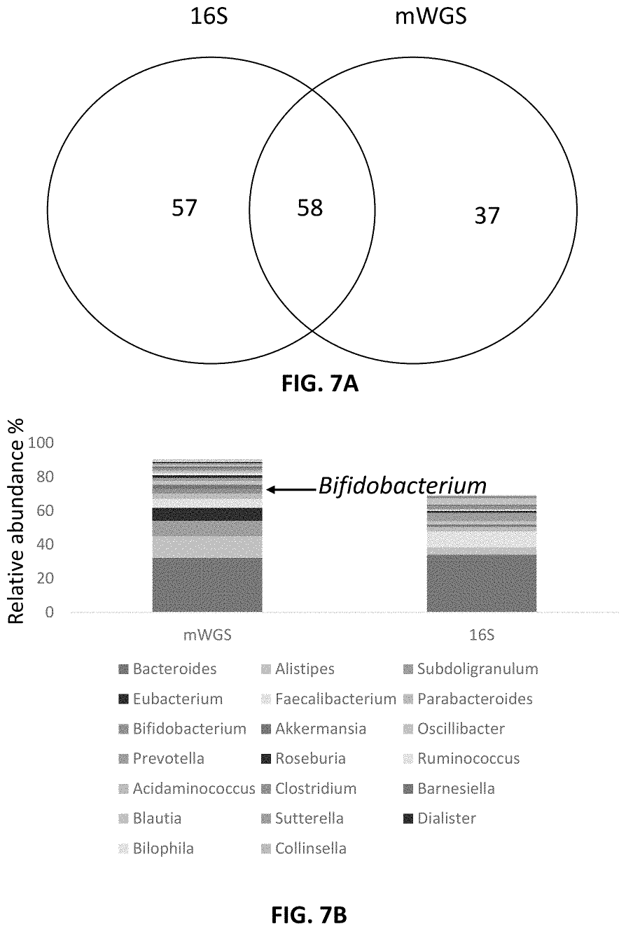

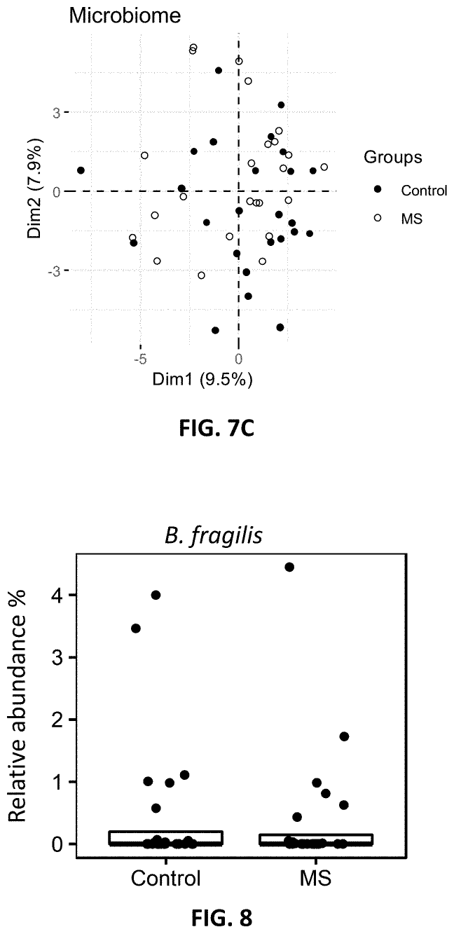

First, gut microbiome profiles at baseline in our MS and control groups were compared using 16S rRNA gene sequencing. Principal component analysis (PCA) plot (FIG. 6A) demonstrated no clustering that distinguished the MS microbiome from that of controls at the operational taxonomic unit (OTU) level. PERMANOVA analysis after adjusting for confounding factors (supplements and tobacco use) further confirmed no statistically significant difference between groups (P=0.18), suggesting similar overall gut bacterial community structures in the two groups. Alpha diversity of the gut microbiome was also similar between MS and controls (data not shown). These stools were then subjected to metagenomic shotgun sequencing (mWGS). mWGS and 16S sequences shared most of the dominant bacteria (58 genera) in samples, except for Bifidobacteria, which was detected only in the mWGS data (FIGS. 7A and B). PCA analysis and PERMANOVA test using mWGS data also failed to identify a distinctive MS microbiome community structure at the species level (P=0.11) (FIG. 7C), corroborating the 16S rRNA gene sequence findings. Next, the variance explained by individual host characteristics and other factors that might influence the microbiome community structure was quantified, in particular diet and host immune response (FIG. 6B). By PERMANOVA, the tested variable explains only a small proportion of microbiome variance. Peripheral blood immune profile and BMI accounted for 3.7% and 2.9% of the total variance in the gut microbiome, respectively, and both effects were statistically significant (P=0.03). Participant status (MS or control), diet, age, race, sex and supplements accounted for small proportions of total microbiome variation (FIG. 6B), but none achieved statistical significance (P=0.18, 0.15, 0.25, 0.25, 0.56, 0.88, respectively). Hence, host immunity and BMI modestly govern the configuration of the gut microbial community in MS and controls, but that other yet-to-be-determined factors play larger roles.

Specific Gut Microbiota Associated with MS and Initiation of DMT Treatment

Next, changes in specific microbes that might be associated with MS were identified. To do so, the gut microbiota composition between MS and controls was compared by differential microbiome abundance analysis using DESeq2 at baseline. 16S rRNA gene sequencing demonstrated that the relative abundances of two Faecalibacterium, one Prevotella, and one Anaerostipes OTU were significantly decreased in MS patients after multiple comparison corrections by false discovery rate (FDR) (FIG. 6C, FDR<0.05). The relative abundances of Prevotella were bimodally distributed, with high abundances in only several healthy controls, in accordance with Human Microbiome Project data, in which only a small fraction of healthy American adults harbor Prevotella in high abundance. mWGS data also identified five species that were significantly lower in abundance in MS patients compared to controls, three of which have known immunomodulatory properties (Bifidobacterium longum, Clostridium leptum, Faecalibacterium prausnitzii) (FIG. 6D, FDR<0.05). The other two unclassified bacterial species from Parabacteriodes and Escherichia, were significantly increased or decreased, respectively, in MS patients compared to controls (FIG. 6D). Thus, a lower abundance of Faecalibacterium species was consistently detected by both sequencing technologies. The average relative abundance of B. fragilis, which is protective in the EAE model, was 0.34% at baseline, and no statistically significant differences between MS and controls were found by univariate analysis (FIG. 8). In summary, decreased relative abundances of bacteria with immunomodulatory properties seem to be the characteristic microbiome change in MS participants vs. controls.

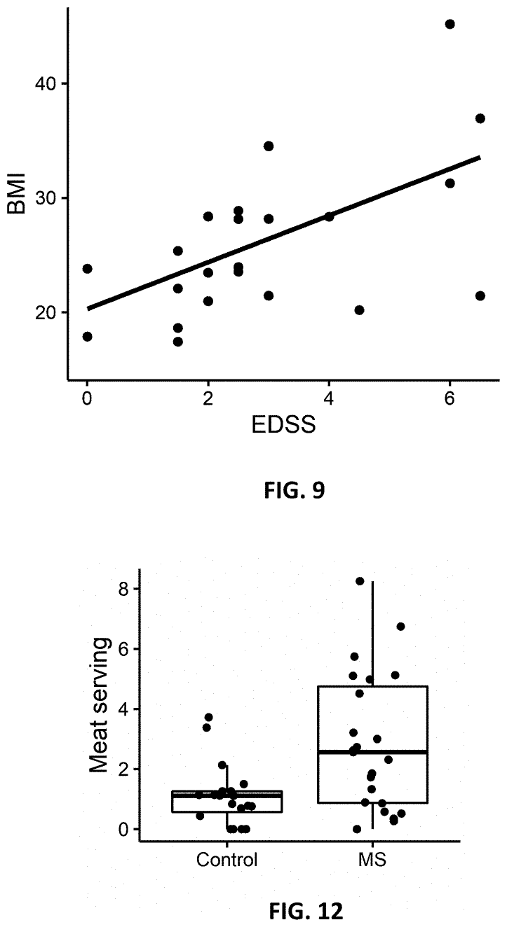

Among MS patients, the gut microbiome differed significantly by the degree of disability at baseline (P=0.03, PERMANOVA), as measured by the expanded disability status scale (EDSS) (mean 2.9, range 0-6.5) (P=0.03, PERMANOVA). However, the difference lost statistical significance after controlling for BMI (P=0.20). BMI and EDSS were positively correlated (Pearson correlation r=0.56, P=0.005) (FIG. 9). Hence, the data do not suggest that EDSS is independently associated with gut bacterial content in MS.

No MS patient had received DMT for at least 3 months before study entry, but in the following six months 11 out of 30 MS subjects (36.7%) initiated DMT. Using DESeq2, it was determined that participants who initiated DMTs within the subsequent six months had, at baseline, significantly lower abundance of Roseburia (FDR=0.03) and higher abundances of Barnesiella (FDR=0.04) and Bacteroides (FDR=0.0004) (FIG. 6E) than those who remained DMT-naïve. MS patients who had a higher relative abundance of Barnesiella were more likely to initiate DMT within six months (OR=2.5, 95% CI 1.3-7.8, P=0.04). Thus, a microbiome difference existed at study entry between those MS participants who initiated DMT subsequently or those who did not, even though they were similar in EDSS, disease duration, BMI, and other characteristics (Table 1 in APPENDIX C).

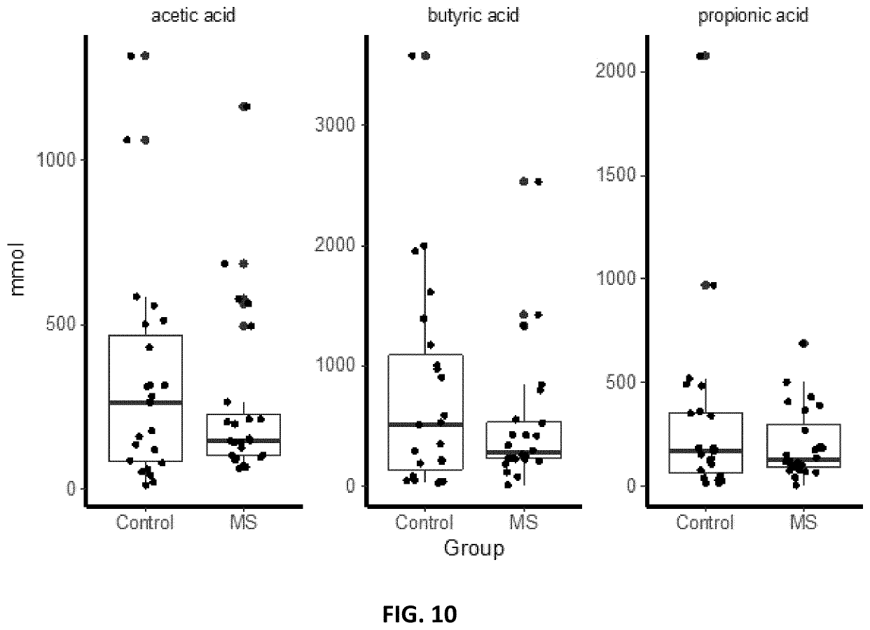

Next, the metabolic potentials of the gut microbiome for all participants were inferred using mWGS data by HUMAnN2 and LEFSe. Sixty-one metabolic pathways and 387 gene Ortholog or KEGG orthologs (KOs) significantly differed between MS cases and controls before adjusting for multiple comparisons (Supplementary Table 1 in APPENDIX C). Interestingly, most differentiating pathways (55/61=90.2%), which included glycolysis, glutamate degradation, fermentation pathways or phospholipid biosynthesis, and KOs (360/387=93.0%), were under-represented in MS cases compared to controls. However, after adjusting for multiple comparisons, no KO or pathway differed significantly between the two groups (all FDR>0.3). Additionally, concentrations of short-chain fatty acids (SCFAs) acetic acid, and butyric acid in stool as determined by GC-MS were moderately lower in the stools of MS patients than in those of controls (FIG. 10), but the trend did not attain statistical significance (P>0.05, Wilcoxon Rank Sum Test). Overall, our data indicate a general pattern of reduction of the gut metabolic potential and SCFAs in MS patients compared to controls.

Loss of the Microbiome-Immune Homeostasis and Establishment of an Immune-Metabolome Association in MS

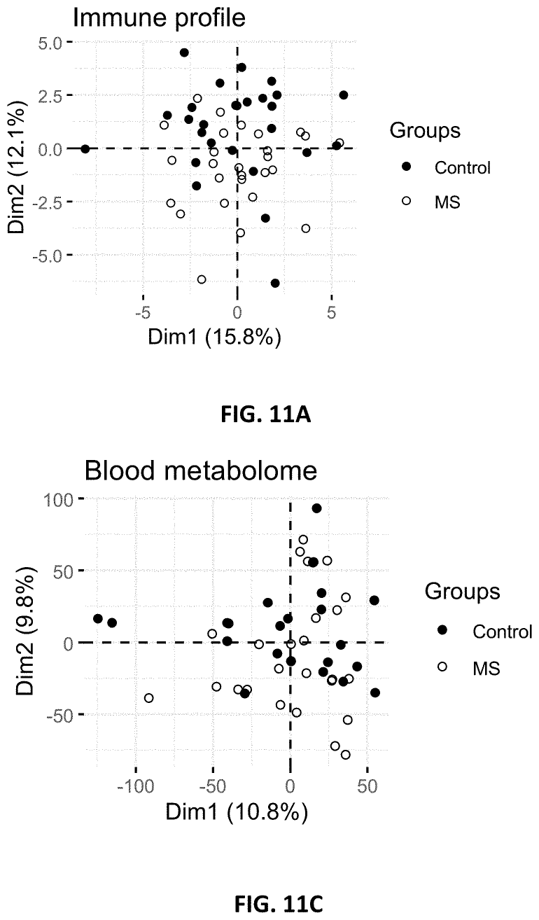

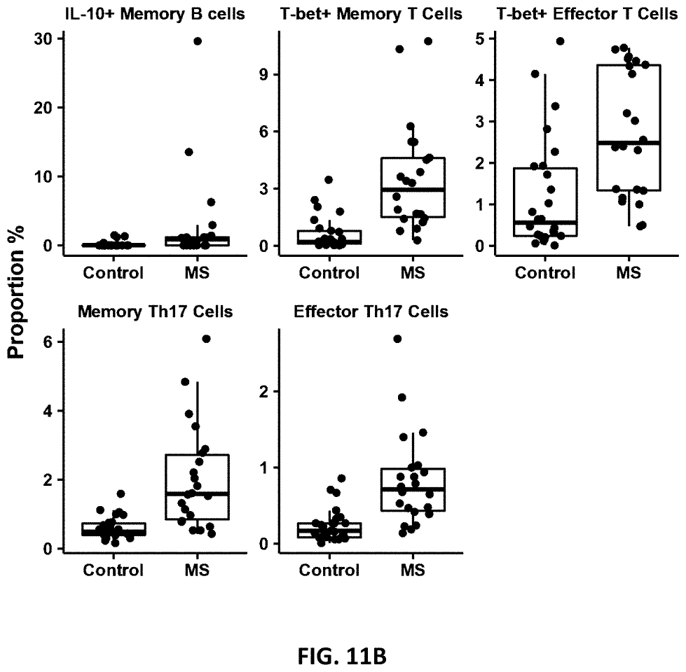

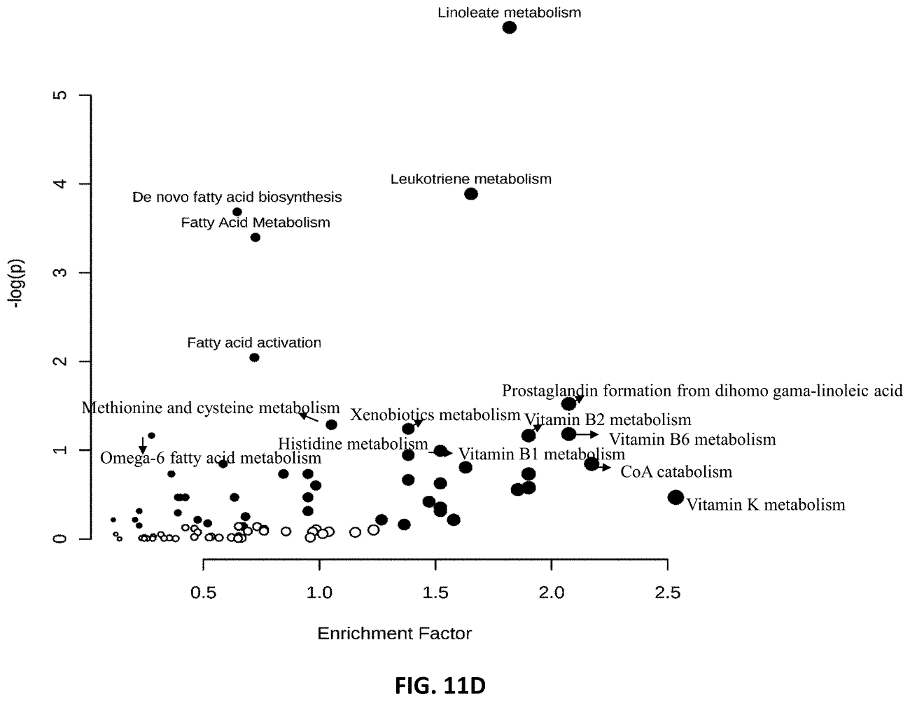

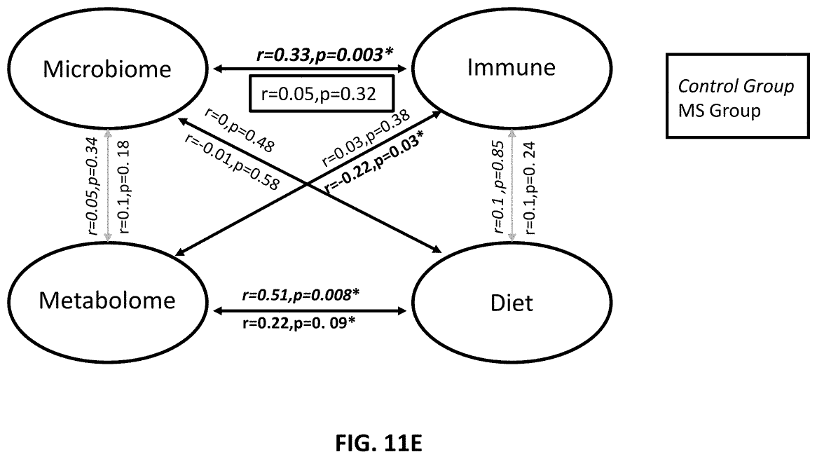

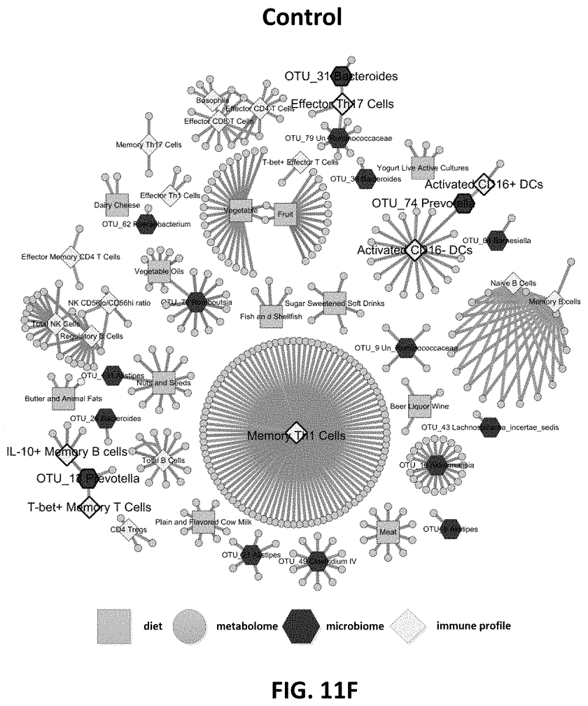

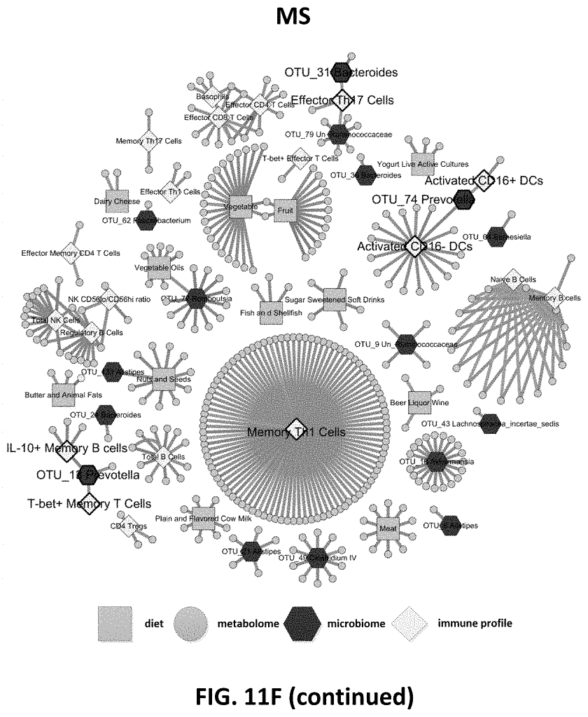

Then, the extended gut microbiome associated with peripheral blood immune and metabolome profiles was interrogated. PCA analysis of 42 blood immune cell populations and intracellular cytokines at baseline indicated an overall significant difference between the MS and controls (P=0.02, PERMANOVA, FIG. 11A), which clustered separately. Specific immune cell subsets analysis by Wilcoxon Rank Sum tests after multiple comparison correction showed that the percentages of peripheral blood IL-10+ memory B cells, T-bet+ memory and effector T cells, memory and effector Th17 cells were significantly greater in MS than in controls (FIG. 11B; Table 2 in APPENDIX C), suggesting an overactive peripheral pro-inflammatory response in untreated MS patients. No significant differences in B and T regulatory immune cells between MS and controls were found. Untargeted metabolomics analysis of serum from MS and controls at baseline identified 4966 potential metabolites, but no clustering in the PCA plot of the metabolites distinguished MS patients from controls (P=0.18, PERMANOVA, FIG. 11C). However, 173 metabolites were differentially represented in MS and controls by Welch's t-test after correcting for multiple comparison by FDR (FDR<0.05). Interestingly, the preponderance of these metabolites was significantly greater in MS patients than in controls. As expected, most metabolites were not annotated in reference databases, but among those that were, plant-associated metabolites such as dihydrochalcone, 9,12-hexadecadienoic acid, and chalcone enriched in the MS patients were found. Pathway analysis showed that pathways involved in linoleate metabolism, fatty acid metabolism, and leukotriene metabolism were altered in MS patients (FIG. 11D). Overall dietary patterns did not differ significantly by PERMANOVA between MS patients and controls. The MS group had a higher median meat intake compared to controls before (P=0.01, Wilcoxon Rank Sum Test) (FIG. 12), but not after FDR adjustment (FDR=0.20).

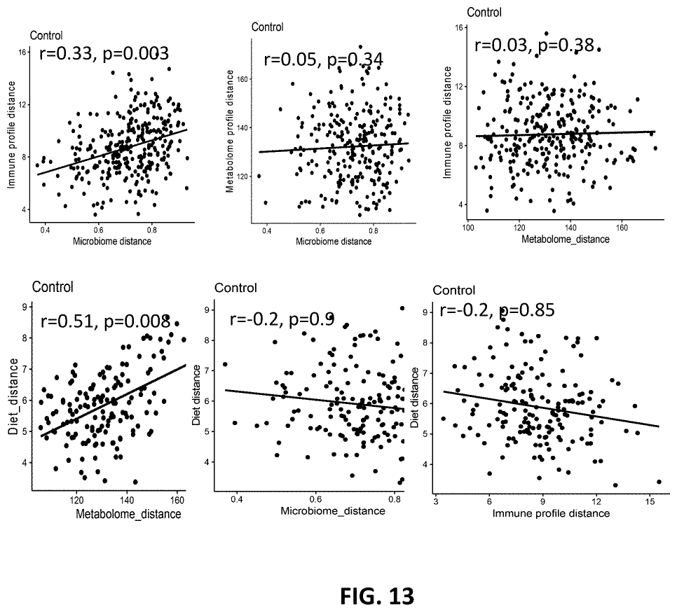

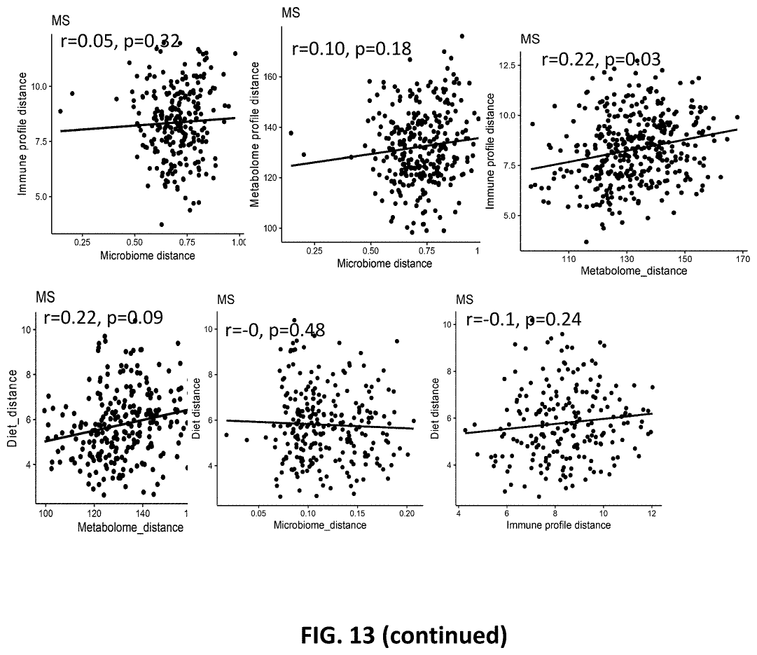

Next, correlations between the gut microbiome, peripheral immune and blood metabolome profiles, and diet were sought, in MS and controls at baseline. The gut microbiome and host blood immune profile were positively correlated in controls (r=0.33, P=0.003, Mantel test) (FIG. 11E, FIG. 13), suggesting a close interaction between the gut microbiome and peripheral immune profiles in individuals without MS. However, this association was absent in MS patients (r=0.05, P=0.32, Mantel Test), suggesting this disease was associated with a disrupted immune-microbiome homeostatic relationship. Strikingly, a positive correlation between peripheral immune and metabolome profiles in MS patients was found (r=0.22, P=0.03, Mantel test), but not in controls (r=0.03, P=0.38). The association between immune and metabolome profiles signifies potentially concomitant changes in blood immune cell populations and metabolism in MS patients that are not observed in healthy controls. In addition, the diet was positively correlated with the blood metabolome in controls (r=0.55, P=0.008, Mantel Test), an association that was partly maintained in MS patients (r=0.22, P=0.09, Mantel Test), suggesting that routine dietary intakes profoundly influence the blood metabolome in health and disease status. Significant associations between diet and the gut microbiome or the peripheral immune profile by Mantel correlations in either MS patients, controls, or the two groups combined were not found. Taken together, our multi-OMICS analysis demonstrates a disruption of the gut microbiome-immune homeostatic relationship in MS participants and positive interaction between immune and metabolome in MS.

Next, a large-scale association analysis to identify specific correlated features within and between OMICS datasets by Pearson correlation was performed (Supplementary Table 4 in APPENDIX C). Within and between group comparisons contained 222 and 384 significant correlations for MS patients and controls, respectively (Supplementary Table 4 in APPENDIX C). Strong and significant correlations (FDR<0.05 for the metabolome and FDR<0.2 for other OMICS, r>0.7 or r←0.7) are presented as complex networks in FIG. 11F. Specifically, in controls, OTU_13_Prevotella (P. copri, lower left in FIG. 11F) was strongly and positively correlated with circulating proportions of IL-10+ memory B cells and T-bet+ memory T cells proportions in the circulation. OTU_74_Prevotella (P. stercorea, top right) was positively correlated with activated CD16+ dendritic cells. OUT 31 Bacteroides (B. coprocol, top) were also positively correlated with effector Th17 cells. In addition, we identified a correlation hub with memory Th1 cells being the node connecting to a variety of blood metabolites. In contrast to controls, in stools from MS participants, OTU_13_P. copri formed a hub that was strongly correlated with blood metabolites in MS, but was not significantly correlated with any circulating immune cell populations. Another particularly notable gut microbiota and blood metabolite hub identified in MS participants involved OUT_2_Bacteroides (B. uniform is, in the center), a highly abundant and prevalent human gut bacterium. Interestingly, we also identified a tandem and positive association between sugar-sweetened soft drinks with effector Th17 cells (top), and the latter was further correlated with a blood metabolite. This finding is consistent with the aggravation of EAE by increased Th17 cells following long-term consumption of high sucrose content beverages. Together, the data infer distinct, diverse, and cross-system interrelationships of key molecules of MS patients and controls, providing a compendium of potential targets for future studies of pathogenic mechanisms underlying MS.

Host-Microbiome Multi-OMICS in Classification of MS Patients and Controls

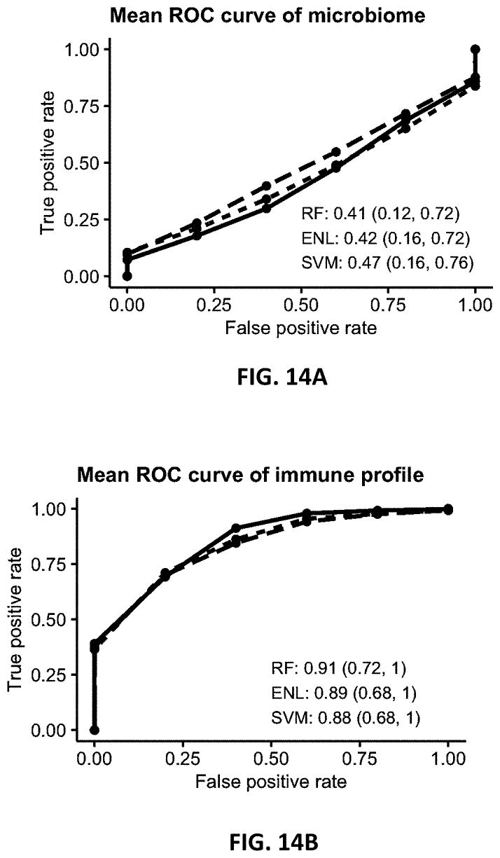

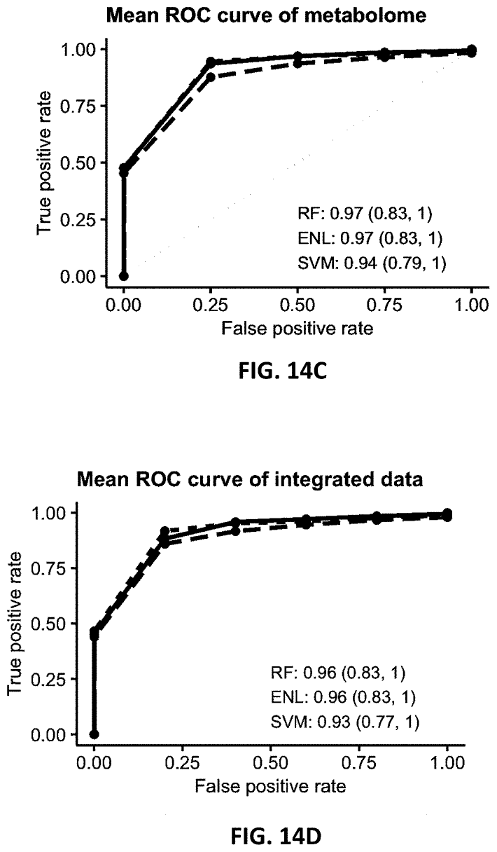

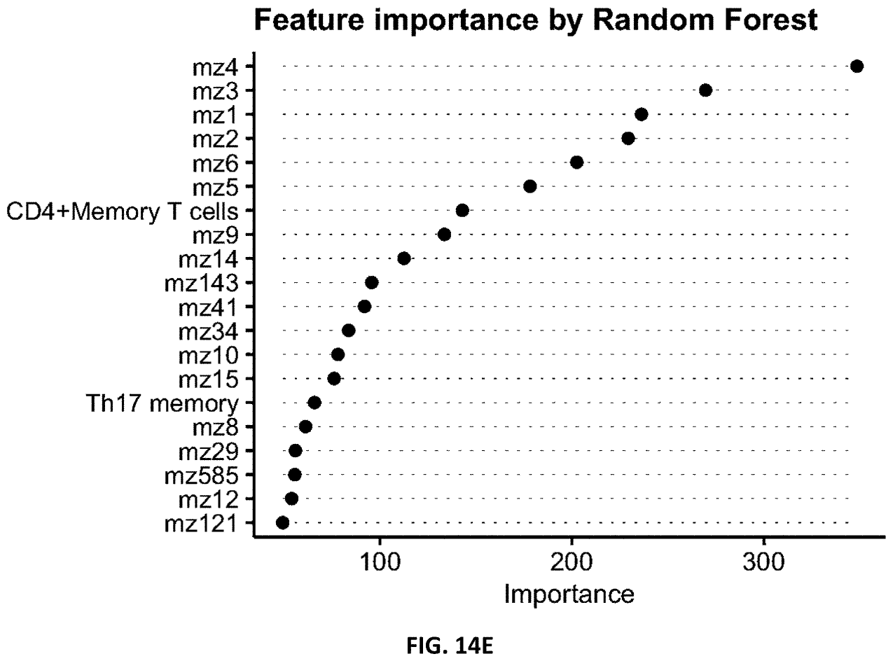

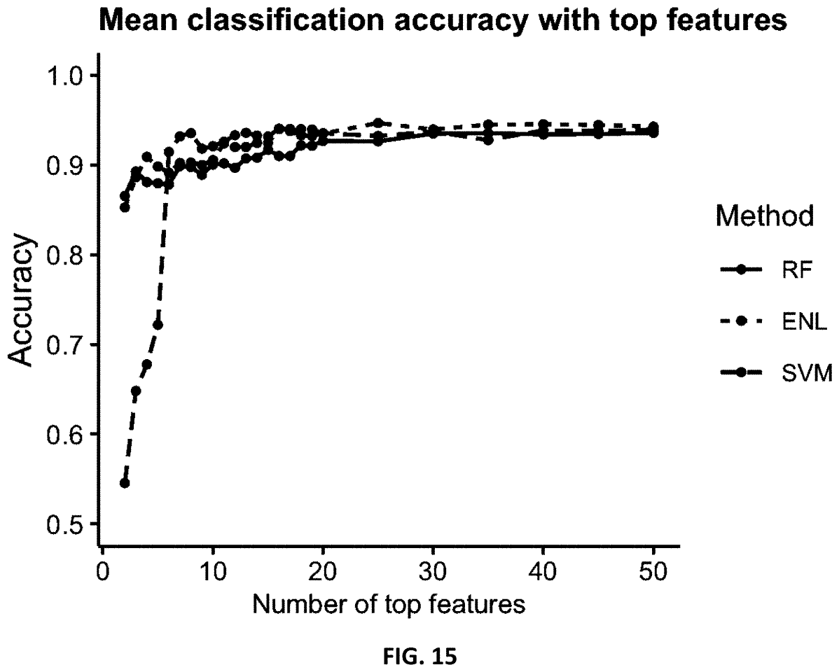

To investigate the power of individual and multi-OMICS to classify MS patients and controls, random forest (RF), elastic net regularized linear regression (ENL) and elastic net regularized support vector machine (SVM), which are suited for high dimension data, were applied. The three classifiers constructed based on blood metabolome and immune profile had the greatest out-of-sample classification performance, with mean Area Under the Curve (AUC) close to, or exceeding 0.90 (FIGS. 14B and C). By contrast, the classification of MS patients and controls based on either all or a subset of microbiome features after marginal screening generated an AUC with a wider range (FIG. 14A), indicating unstable classification performance using the gut microbiome alone. To determine if the integration of data types improved classification performance, three classification models with all the features of the microbiome, blood metabolome, and immune profile were trained. The mean AUC obtained with integrated data was comparable to that obtained from the blood metabolome data (FIG. 14D). The three classifiers trained with blood metabolome, immune profile, or integrated data performed similarly, though RF performed best in classifying MS patients and controls. To identify the top predictive features, the feature importance measures from the RF model trained with the integrated data (averaged over 200 random splits) were examined. As expected, the top 20 variable-of-importance consisted of blood metabolites and immune cell populations, such as T-bet+ memory T cells and memory Th17 Cells (FIG. 14E). Notably, these highest-ranking features achieved similar classification accuracy as using all the OMICS features (FIG. 15).

Longitudinal Changes of the Gut Microbiome and Host Peripheral Immune and Metabolome Profiles in MS Patients and Controls

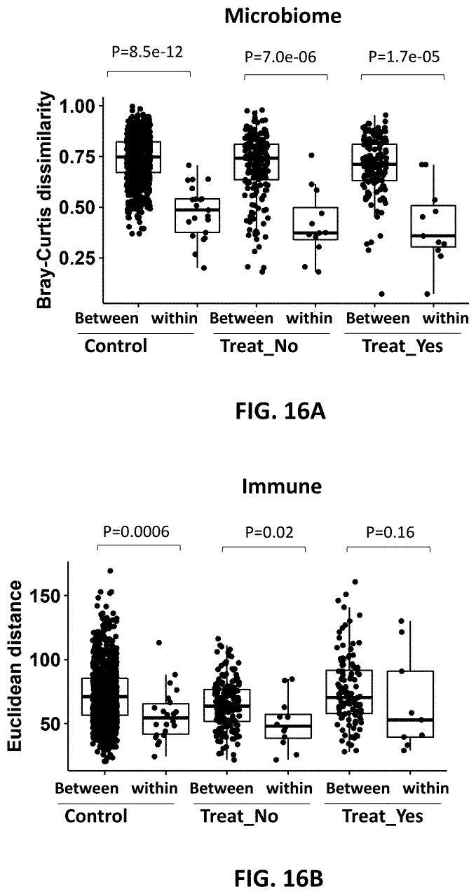

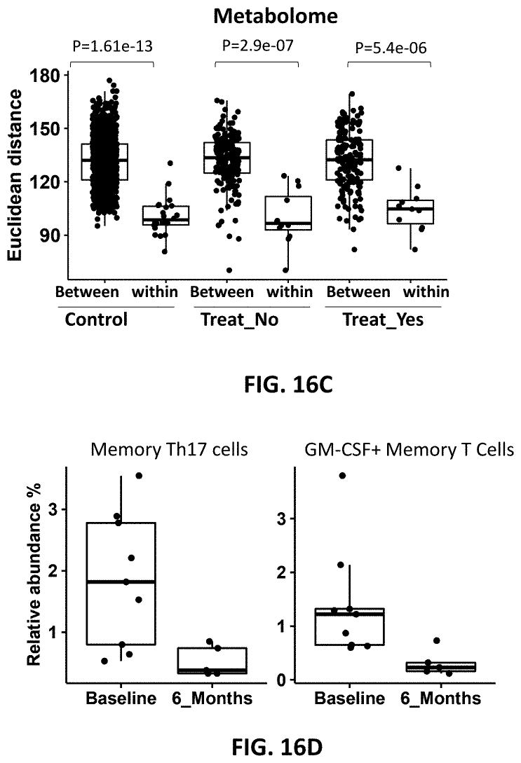

To first measure the temporal stability of each OMICS over time (6 months), the pair-wised dissimilarity between and within the controls, MS patients who did and did not receive DMTs, were computed. Within- and between-participant dissimilarity refers to the dissimilarity of baseline and six months for the same individuals, and dissimilarity between different individuals at each time point, respectively; smaller within-participant dissimilarity infers temporal stability relatively to between-participant dissimilarity. Compared to between-participant variation, within-participant variations of the microbiome and metabolome were significantly lower for all MS patients and controls (FIGS. 16A and C), and the within-participant variations of the immune profile were also significantly lower in controls (FIG. 16B). This suggests a relatively stable overall microbiome and metabolome for MS patients and controls as well as the immune profile in controls during the study period. In contrast, between- and within-participant variations of the blood immune profile in MS patients who received treatment with DMTs were at a similar level (P=0.16, Wilcoxon-rank test, FIG. 16B). The non-significance of between- and within-participant variations for DMT treated patients suggests a greater change of the immune profile in treated MS patients. Wilcoxon Rank Sum test showed that the proportions of memory Th17 cells and GM-CSF+ memory T Cells were significantly reduced at 6 months compared to baseline in MS patients who initiated DMTs (FDR=0.05, FIG. 16D). Further, a Mantel correlation was performed to test whether or not between-participant similarities are maintained over the study interval based on distance measures of the gut microbiome, blood immune cell, and metabolome profiles. Significant correlation of the gut microbiome (r=0.3, P=0.001 for MS; r=0.5, P=0.001 for controls) and blood metabolome (r=0.3, p=0.003 for MS; r=0.3, p=0.003 for controls) between baseline and six-month samples was found. In contrast, the blood immune profile showed no correlation between baseline and six-month for MS patients (r=0.05, P=0.33), while the controls demonstrated significant correlation (r=0.33, p=0.01). These findings suggest that between-participant dissimilarity was maintained over the study course in microbiome and metabolome, but not in the immune profiles. Specific gut microbiome, metabolome, or food serving that significantly changed between study entry and six-month follow-up in the MS patients were not identified. Microbiota that differed between treated and untreated MS patients at six months were also not identified. Strikingly, 77.6% of the significantly differing blood metabolites between MS patients and controls at baseline maintained these differences at six months. The remaining metabolites were also preserved at similar levels, but a non-statistical difference was found between the two groups. The machine learning models constructed based on baseline metabolome features consistently showed the best classification accuracy in differentiating MS patients from controls at six months, compared to those constructed based on immune and microbiome profiles. In summary, the host peripheral immune profile, blood metabolites and the gut microbiome of MS participants remain relatively stable over 6 months in MS patients who remained untreated by DMT, while treatment with DMTs affected specific immune cell populations.

Materials and Methods

Study Participants

This prospective case-control cohort study was approved by the Human Research Protection Office at Washington University in St. Louis School of Medicine (WUSM) (approval number: 201502105). MS patients were consecutively recruited at the John L. Trotter MS Center of WUSM. Inclusion criteria for MS patients were: (1) diagnosis of MS using the 2010 revision of the McDonald criteria 39; (2) no DMT or steroid treatments in the past 3 months; (3) ages 18 to 50 years; and 4) not in clinical relapse at study enrollment. Exclusion criteria were: (1) coexistence of other chronic inflammatory (e.g. asthma, chronic hepatitis, inflammatory bowel disease, celiac disease, etc.) and autoimmune (e.g. rheumatoid arthritis, SLE, type I diabetes, etc.), or metabolic (e.g. type II diabetes, familial hypercholesterolemia, etc.) diseases. (2) Antibiotics or steroid therapy in the past 3 months. (3) History of immunosuppressive or chemotherapeutic treatment, (4) history of chronic infectious disease (e.g. TBC, HIV, HBV, HCV, etc.). (5) neoplastic disease not in complete remission, and (6) pregnancy. Age, gender, BMI, and ethnicity matched healthy controls were enrolled using the same exclusion criteria. Table 1 in APPENDIX C details case and control demographic and clinical characteristics at enrollment. MS participants and controls were followed up at six months after enrollment. Although DMT commencement was strongly recommended to the 30 MS patients by their clinicians, only 11 received DMT during the six-month study period. The two main reasons for not starting treatment within the 6 months duration of this study were administrative delays in obtaining approvals and patient choice. The DMTs started were natalizumab and rituximab (n=1 each), glatiramer acetate, fingolimod, interferon-β1a (n=2 each), and dymetilfumarate (n=3).

Sample Collection

The stool and blood of all participants were collected at the time of enrollment and six months later. Stools were self-collected and placed on frozen gel packs and shipped overnight to the research laboratory. Upon receipt, stools were immediately stored at −80 C until further processing. Stools from baseline and six months were processed at the same time for DNA extraction and microbiome sequencing to minimize batch effects among the specimens. Blood was collected in heparinized tubes, insulated, and shipped at room temperature overnight to Ohio State University for immunophenotyping. Peripheral blood mononuclear cells (PBMCs) were isolated immediately on arrival and analyzed by flow cytometry. Stool DNA extraction and microbiome sequencing 16S rRNA gene sequencing permits deep microbiota profiling, especially of low abundance taxa. Metagenomic whole genome shotgun sequencing (mWGS) provides classification to species levels but may not enumerate low abundance bacteria. We applied these complementary platforms to sequencing platforms for the gut microbiome characterization. Stool DNA extraction and sequencing were performed as we have done previously. In brief, stool DNAs were extracted using the MOBIO PowerSoil DNA Extraction kit. For 16S rRNA gene sequencing, hyper-variable regions V1-V3 of the 16S gene were amplified using primers 27F and 534R (27F:5′-AGAGTTTGATCCTGGCTCAG-3′ (SEQ ID NO:1) and 534R: 5′-ATTACCGCGGCTGCTGG-3′ (SEQ ID NO:2)). 16S libraries were prepared and sequenced on the Illumina MiSeq sequencing platform using a V3 2×300 bp paired-end sequencing protocol with a target read depth of 10,000 reads/sample. Illumina's software handles the initial processing of all the raw sequencing data. One mismatch in primer and zero mismatch in barcodes were applied to sample deconvolution. Reads were further processed by removing sequences with low quality (average qual 20% in QC samples was excluded to guarantee the quality of the data set, followed by the univariate and multivariate analysis to differentiate the unbiased metabolites. The resulting m/z values were subjected to the “MS peaks to pathways” analysis in Metaboanalyst (https://www.metaboanalyst.ca/) to analyze pathways and identify metabolites with a maximum error of 5 ppm using KEGG and Metlin databases. Welch's t-test was used to determine significant changes between the control and MS groups. Previous studies supported that parametric and non-parametric univariate tests result in very similar results for metabolome data. P values were further adjusted by the FDR approach.

Mantel Correlation and Multi-OMIC Feature-Feature Correlation

Covariation between multi-OMICS using Mantel tests (Pearson correlation between distances of two matrices) was quantified. A pair-wised inter-participant variation/distance matrix was first computed for each OMIC dataset, with Bray-Curtis dissimilarity for the microbiome data and Euclidean distance for the immune profile, blood metabolome, and food intakes. Inter-participant dissimilarity matrices were then compared using the mantel function in the vegan package. Mantel correlation analysis was also conducted similarly to quantify longitudinal covariation for two given OMICS data. The significance of the statistic is produced by permuting rows and columns of the first dissimilarity matrix 1000 times. Feature-feature correlations within and between OMICS datasets using Pearson correlation with cor.test function in the stats package in R were performed. Because of the potential for different interactions in MS patients and controls, all correlations were performed separately for the two groups, accounting for BMI and age. P values were corrected based on FDR approach. FDR 0.7 were considered strong correlations and illustrated using Cytoscape. A hub in the correlation network was defined as nodes with at least 20 connections. All correlation results including before and after FDR corrections and after manual inspections are summarized in Table S4 in APPENDIX C. MS classification using machine learning models We tested three machine learning models (random forest (RF), elastic net regularized linear regression (ENL), and elastic net regularized support vector machine (SVM)) to classify MS patients and controls. All three models can be used to analyze high-dimensional data (when the number of features is larger than the sample size) and to generate measures of feature importance. The models were trained by each individual OMICS to determine the importance of a given OMICS data in classification performance (FIGS. 14A, B, and C), or by the combination of all the OMICS to determine whether it achieves a better classification performance (FIG. 14D). For the microbiome data, OTU counts were converted to compositions, in which zeros were replaced by 0.5, the maximum rounding error. The centered log-ratio transformation was then applied so that the transformed data obeyed the Euclidean geometry. The number of raw features could greatly exceed the sample size and many of them are either redundant or irrelevant in distinguishing MS patients and controls. Hence, the size of the feature set was reduced before fitting any machine learning model, by applying a statistical marginal screening procedure through multiple hypothesis testing with false discovery rate control. Such a hybrid “marginal screening+machine learning” approach facilitated the model training and consistently improved the performance of the classifiers in our study. The data were randomly divided into a training set with 80% samples to build a classifier and a testing set with the remaining 20% of the samples to evaluate the performance of the resulting classifier. Under each setting, this random-splitting procedure was repeated 200 times to stably assess the out-of-sample predictability of a classifier and its associated feature importance. Specifically, in each random split, when training the ENL and the SVM, we used Leave-one-out Cross Validation (LOOCV) to tune their regularization parameters. For RF, we set the maximum number of features allowed to try in an individual tree as the square root of the number of features, and the number of trees as 30,000, a sufficiently large number. The feature importance in the RF is measured by the Mean Decrease Gini. After building a classifier using the training samples, the testing samples were used to compute its pairs of out-of-sample true positive rate (TPR) and false positive rate (FPR), based on which the sample receiver operating characteristic (ROC) curve was constructed and calculated the corresponding area under the ROC curve (AUC) value. By aggregating these results from 200 random splits, we draw the average ROC curve and computed the average AUC and its 90% confidence interval for each classifier.

What is claimed is:

1. A method for identifying MS in a subject, the method comprising:
obtaining a blood sample and a fecal sample from the subject;
determining an immune profile based on the blood sample;
determining a gut microbiome profile based on the fecal sample;
determining a strength of an immune-microbial homeostatic relationship based on the immune profile and the gut microbiome profile; and
identifying MS in the subject if the strength of an immune-microbial homeostatic relationship falls below a threshold value.

2. The method of claim 1, further comprising defining the threshold value based on a comparison of a first plurality of control strengths of immune-microbial homeostatic relationships of healthy controls and a second plurality of control strengths of immune-microbial homeostatic relationships of known MS patients.

3. A method of identifying an MS patient likely to seek disease-modifying treatment within six months, the method comprising:
obtaining a fecal sample from the subject,
determining a gut microbiome profile based on the fecal sample;
determining a relative abundance of Barnesiella spp. based on the gut microbiome profile; and
identifying an MS patient as likely to seek disease-modifying treatment within six months if relative abundance of Barnesiella spp. falls above a threshold value.

4. The method of claim 3, further comprising defining the threshold value based on a comparison of a first plurality of relative abundance of Barnesiella spp. of MS patients known to remain untreated for six months and a second plurality of relative abundance of Barnesiella spp. of MS patients known to seek disease-modifying treatment within six months.
